(19) United States
(12) Patent Application Publication
(10) Pub. No.: US 2007/0184514 A1
(43) Pub. Date: Aug. 9, 2007

(54) METHODS AND COMPOSITIONS FOR DETECTING ACTIVE COMPONENTS USING BIOLUMINESCENT BACTERIA AND THIN-LAYER CHROMATOGRAPHY

(76) Inventors: Sheryl M. Verbitsky, Arvada, CO (US); James D. McChesney, Etta, MS (US); Gerald T. Gourdin, Boulder, CO (US); Larissa M. Ikenouye, Boulder, CO (US)

Correspondence Address: NEEDLE & ROSENBERG, P.C., SUITE 1000, 999 PEACHTREE STREET, ATLANTA, GA, 30309-3915, US

(21) Appl. No.: 11/623,620

(22) Filed: Jan. 16, 2007

Related U.S. Application Data
(60) Provisional application No. 60/766,362, filed on Jan. 13, 2006.

(51) Int. Cl. C12Q 1/18; C12Q 1/04; C12M 3/00

(52) U.S. Cl. 435/32; 435/34; 435/287.1

(57) ABSTRACT

Disclosed are methods and compositions for the detection of compounds.

### CROSS-REFERENCE TO RELATED APPLICATIONS

This application claims benefit of U.S. Provisional Application No. 60/766,362, filed Jan. 13, 2006. Application No. 60/766,362, filed Jan. 13, 2006, is hereby incorporated herein by reference in its entirety.

### STATEMENT REGARDING FEDERALLY SPONSORED RESEARCH

This invention was made with government support under Grant FD-U-002514-01 awarded by the U.S. Food and Drug Administration's Center for Food Safety and Applied Nutrition. The government has certain rights in the invention.

### FIELD OF THE INVENTION

The disclosed invention is generally in the field of detection of compounds and specifically in the area of detection of compounds using bioluminescent bacteria.

### BACKGROUND

Contact bioautography was first published in 1946 when Goodal et al. placed a developed paper chromatogram onto inoculated agar [1]. In 1961 the paper chromatogram was replaced with thin-layer chromatography [2, 3]. In contact bioautography compounds diffuse from the chromatogram to an inoculated agar plate. There are several disadvantages associated with this procedure. For example many compounds can bind irreversibly to the matrix especially silica based matrices and thus some compounds may never diffuse to the agar. Additionally, there are inherent difficulties obtaining adequate contact between the matrix and the agar and often the matrix adheres to the surface [4].

To overcome some of the issues with contact bioautography, direct bioautography was developed. In direct bioautography the chromatogram is dipped into a broth of microorganisms. The microorganisms are subsequently cultured directly on the chromatogram and results visualized using tetrazolium dyes [5]. A commercialized version of this method, Chrom Biodip® Antibiotics has been developed and is available through Merck KGaA (Darmstadt, Germany). It uses a bacterial solution of Bacillus subtilis followed by over night incubation and spraying with a MTT-tetrazolium salt visualization reagent [6]. These methods also face a variety of limitations. For example, the bacterial solution can only be applied to one plate followed by a long incubation period. Additionally, a visualization agent is required to detect results.

Many of these problems have been addressed through the direct coupling of paper or thin-layer chromatography with luminescent bacteria [7-12]. Most notably, these methods eliminate the need for a secondary detection reagent by utilizing the inherent luminescent properties of the luminescent bacteria. Secondly, the luminescent bacteria can be liquid cultured prior to plate application thus permitting multiple plate applications from one culture. Despite these improvements there are many areas where these early methods need to be revamped to produce adequate results. This present invention provides the advancements that greatly enhance data quality, permits acid and base usage, alternative detection methods, and kit compatibility.

### BRIEF SUMMARY

The disclosed methods are screening methods, including rapid screening methods, for complex mixtures such as dietary supplements, food stuffs, waste water, natural products, etc. This methodology can detect discrete components of complex mixtures that act as toxins. This assay can be used to support material identity, detect toxins and chemical adulterants, and control manufacturing procedures. This technology is kit compatible thus providing a rapid and inexpensive analysis of many complex samples.

Some useful forms of the disclosed method are compatible with the use of a wide variety of chromatographic solvents and acids and bases used in chromatographic solvents. It has been discovered that use of certain buffers and pH adjusting substances, such as HEPES buffer, with the luminescent bacteria eliminates or reduces negative effects chromatographic solvents and acids and bases used in chromatographic solvents would otherwise have on the luminescent bacteria. The use of such buffers and pH adjusting compounds allows the disclosed methods and compositions to use virtually any desired chromatographic solvent or acid or base used in chromatographic solvents.

Some useful forms of the disclosed methods use a “squeegee” effect, such as by use of a squeegee device, to remove excess bacteria from the chromatographic matrix. It has been discovered that removal of excess bacteria from the chromatographic matrix provides more reliable and consistent results and readouts of luminescence in the disclosed methods. It is believed that use of the squeegee effect or squeegee device produces a more even and/or thinner layer of bacteria on the chromatographic matrix. Use of a squeegee effect or squeegee device also allows the chromatographic matrix to be applied to the matrix by immersion of the matrix in a bacterial suspension. This greatly simplified preparation of bacteria to be used in the method since a simple bacterial suspension can be used and since such a suspension can be applied by immersion. The use of a squeegee effect or squeegee device allows such immersion application of the bacteria to produce more reliable and consistent results and readouts of luminescence.

Disclosed is a method comprising bringing into contact luminescent bacteria, a pH adjusting component and a thin-layer chromatography matrix, and detecting inhibited luminescence. The chromatography matrix comprises a sample separated by thin-layer chromatography.

Also disclosed is a method comprising bringing into contact luminescent bacteria and a thin-layer chromatography matrix, and detecting inhibited luminescence. The chromatography matrix comprises a sample separated by thin-layer chromatography, the luminescent bacteria are brought into contact with the chromatography matrix by applying the bacteria to the chromatography matrix and using a squeegee effect to remove excess bacteria from the chromatography matrix.

The luminescent bacteria can be brought into contact with the chromatography matrix by applying the bacteria to the chromatography matrix and using a squeegee effect to remove excess bacteria from the chromatography matrix. The squeegee effect can be achieved using a squeegee device. The bacteria can be applied by immersing the chromatography matrix is a liquid comprising the bacteria. The liquid can further comprise the pH adjusting compound. The pH adjusting component can be a buffer. The buffer can buffer in the 7.5±1 pH range. The buffer can be in the 0.2-0.5 M concentration range. The buffer can be HEPES [N-(2-Hydroxyethyl)piperazine-N′-(2-ethanesulfonic acid)] buffer. The buffer can be Tris(hydroxymethyl)aminomethane buffer.

The luminescence inhibition can be recorded with x-ray or Polaroid film, cooled CCD camera, video imaging, 35 mm film, or Polaroid photo documentation system. The luminescent bacteria can comprise Vibrio fischeri. The luminescent bacteria can be cultured prior to being brought into contact with the chromatography matrix. The luminescent bacteria can be stored in lyophilized form prior to being cultured. The sample can comprise dietary supplements, natural products, foodstuffs, beverages, waste water, soil samples, pharmaceuticals, pesticides, herbicides, fungicides, insecticides, heavy metals, or a combination. Detection of inhibited luminescence indicates the presence of an active compound in the chromatography matrix at the site of the inhibited luminescence.

Also disclosed is a kit comprising luminescent bacteria and a pH adjusting component. The kit can further comprise a thin-layer chromatography matrix. The kit can further comprise a squeegee device. The kit can further comprise media culture materials. The media culture materials can be stored as a combined dry form. The luminescent bacteria can be stabilized by lyophilization in the presence of sucrose at a ratio of 0.2 g±0.1 sucrose/mL. The pH adjusting component can be a buffer. The buffer can buffer in the 7.5±1 pH range. The buffer can be in the 0.2-0.5 M concentration range. The buffer can be HEPES [N-(2-Hydroxyethyl)piperazine-N′-(2-ethanesulfonic acid)] buffer. The buffer can be Tris(hydroxymethyl)aminomethane buffer.

Also disclosed is a biosensor/bioreporter technology that separates mixtures into separate components by high performance thin-layer chromatography (HPTLC), directly contacts bioluminescent bacteria to chromatography plate, and detects inhibited luminescence. Also disclosed is a biosensor/bioreporter kit that separates mixtures into separate components by high performance thin-layer chromatography (HPTLC), directly contacts bioluminescent bacteria to chromatography plate, and detects inhibited luminescence.

The mixtures can include mixtures comprising dietary supplements, natural products, foodstuffs, beverages, waste water, soil samples, pharmaceuticals, pesticides, herbicides, fungicides, insecticides, and heavy metals. The bioluminescent bacteria can be Vibrio fischeri. The luminescence inhibition can be recorded with x-ray or Polaroid film, cooled CCD camera, video imaging, 35 mm film, or Polaroid photo documentation system. The technology can use a buffering system that buffers in the 7.5±1 pH range. The technology can use HEPES [N-(2-Hydroxyethyl)piperazine-N′-(2-ethanesulfonic acid)] buffer in the 0.2-0.5 M range. The technology can use Tris(hydroxymethyl)aminomethane buffer in the 0.2-0.5 M range. The technology can use a squeegee system to remove excess bacteria.

The media culture materials (salts or biologicals) can be stored as a combined dry form. The stabilizing agent used to lyophilize Vibrio fischeri can be sucrose at a ratio of 0.2 g+0.1 sucrose/mL. The bioluminescent bacteria can be cultured or subcultured using a stir bar and stir plate. The bioluminescent bacteria can be cultured or subcultured using a tissue culture flask. The bacteria can be made viable for greater than 2 hours by aspirating air into the culture at least every 15 minutes.

Also disclosed are biosensor assays and kits for analyzing single components or complex mixtures.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows TLC-bioluminescence detected with x-ray film. 72-108³ pmol of 4-nitrophenol (4-np) was applied using the band spray mode of an automatic spotter to an Analtech TLC plate. The TLC plate was coated with luminescent Vibrio fischeri using an automatic immersion device. Kodak X-OMAT LS film was then exposed 5 seconds by direct contact to plastic wrap covering the TLC plate.

FIG. 2 shows HPTLC-bioluminescence detected with Polaroid film. 0.72-108 nmol of 4-nitrophenol (4-np) was applied using the band spray mode of an automatic spotter to an Analtech (top image) TLC and a Merck (bottom image) HPTLC plate. The TLC plates were coated with luminescent Vibrio fischeri using an automatic immersion device. Polapan 672 B&W ISO 400/27° was exposed 10 seconds by direct contact to plastic wrap covering the TLC plate to obtain the top image. A Polaroid photo documentation camera FB-PDC-35 with Polapan 667 ISO 3000/36° film exposed for 10 minutes was used to obtain bottom image.

FIG. 3 shows TLC-bioluminescence detected with CCD camera cooled to −30° C. from ambient. 0.72-144 nmol of 4-nitrophenol (4-np) was applied using the band spray mode of an automatic spotter to a Merck TLC plate. The TLC plate was coated with luminescent Vibrio fischeri using an automatic immersion device. Image was captured using a 2 minute exposure time at a total plate incubation time of 10 minutes.

FIG. 4 shows HPTLC-bioluminescence detected with CCD camera cooled to −30° C. absolute. 0.72-72 nmol of 4-nitrophenol (4-np) was applied using the band spray mode of an automatic spotter to a Uniplate HPTLC plate. The TLC plate was coated with luminescent Vibrio fischeri using an automatic immersion device. Image was captured using a 2 minute exposure time at a total plate incubation time of 10 minutes.

FIG. 5 shows HPTLC-bioluminescence image of Uncaria tomentosa, caffeine, and ascorbic acid.

FIG. 6 shows quantitative HPTLC-bioluminescence analysis of rhizome extracts of Actaea racemosa (black cohosh) adulterated with various amounts of Actaea pachypoda (white cohosh) leaf extract.

FIG. 7 shows HPTLC-bioluminescence analysis of infant formula spiked with strychnine.

FIG. 8 shows HPTLC-bioluminescence of orange juice spiked with strychnine.

FIG. 9 shows HPTLC-bioluminescence of a cola drink spiked with monofluoroacetate (MFA).

FIG. 10 shows HPTLC-bioluminescence of a steroid, heavy metal, and pesticide mixture in tap water.

### DETAILED DESCRIPTION

The disclosed method and compositions may be understood more readily by reference to the following detailed description of particular embodiments and the Example included therein and to the Figures and their previous and following description.

The disclosed methods are screening methods, including rapid screening methods, for complex mixtures such as dietary supplements, food stuffs, waste water, natural products, etc. This methodology can detect discrete components of complex mixtures that act as toxins. This assay can be used to support material identity, detect toxins and chemical adulterants, and control manufacturing procedures.

The disclosed methods and compositions directly couple luminescence, such as bioluminescence, to thin-layer chromatography (TLC) or high performance thin-layer chromatography (HPTLC). Samples, such as liquid samples or extracts, are applied to the chromatography plate then mixtures are separated by TLC or HPTLC. After solvent removal, such as by evaporation, the chromatography plate is coated with luminescent bacteria, such as bioluminescent bacteria, which identifies toxic compounds (single or multiple, depending on chromatographic resolution) as dark zones on a luminescent background. Results can occur within seconds with typical limits of detection for toxic substances in the picomol range. This technology is kit compatible thus providing a rapid and inexpensive analysis of many complex samples.

Some useful forms of the disclosed method are compatible with the use of a wide variety of chromatographic solvents and acids and bases used in chromatographic solvents. It has been discovered that use of certain buffers and pH adjusting substances, such as HEPES buffer, with the luminescent bacteria eliminates or reduces negative effects chromatographic solvents and acids and bases used in chromatographic solvents would otherwise have on the luminescent bacteria. The use of such buffers and pH adjusting compounds allows the disclosed methods and compositions to use virtually any desired chromatographic solvent or acid or base used in chromatographic solvents. Many chromatographic solvents, acids and bases are known and have been used for thin-layer chromatography. The full range of such chromatographic solvents, acids and bases, and thin-layer chromatographic techniques making use of them, can now be used in and with methods and compositions for detection of compounds using luminescent bacteria as described herein.

Some useful forms of the disclosed methods use a “squeegee” effect, such as by use of a squeegee device, to remove excess bacteria from the chromatographic matrix. It has been discovered that removal of excess bacteria from the chromatographic matrix provides more reliable and consistent results and readouts of luminescence in the disclosed methods. It is believed that use of the squeegee effect or squeegee device produces a more even and/or thinner layer of bacteria on the chromatographic matrix. Use of a squeegee effect or squeegee device also allows the chromatographic matrix to be applied to the matrix by immersion of the matrix in a bacterial suspension. This greatly simplified preparation of bacteria to be used in the method since a simple bacterial suspension can be used and since such a suspension can be applied by immersion. The use of a squeegee effect or squeegee device allows such immersion application of the bacteria to produce more reliable and consistent results and readouts of luminescence.

Previous methods apply bioluminescent bacteria to a separation phase matrix utilizing paper chromatography and/or thin-layer chromatography (TLC) plates as the preferred matrix. These matrices minimize compound separation thus limiting detection levels of separated compounds by bioluminescent inhibition. Examples of some methods using bioluminescent bacteria are European Patent No. EP 0 588 139 B1 to Weisemann et al.; U.S. Pat. No. 6,017,722 to Becvar et al.; U.S. Pat. No. 6,238,928 to Weisemann et al.; U.S. Pat. No. 6,340,572 to Becvar et al.; and U.S. Pat. No. 6,673,563 to Becvar et al., each hereby incorporated by reference in their entirety and specifically for their description of thin-layer chromatography methods and compositions using luminescent bacteria. Features, compositions, and techniques used in known methods can be used, applied and adapted for use in the disclosed methods and compositions.

High performance thin-layer chromatography (HPTLC) plates also use adsorption and capillary action to separate complex samples into discrete components but have an average smaller particle size matrix and narrower particle size distribution than TLC plates thus greatly enhancing resolution. Additionally they consume less solvent and give faster results due to a shorter migration time and distance. The disclosed methods provide maximum compound separation and resolution by employing HPTLC plates as the separation medium.

Bacterial application to the matrix of interest can greatly affect data quality. Previous methods prepare a saline bacterial suspension and spray the suspension onto TLC plates. The preparation of the bacterial suspension is time consuming and the spay application produces inferior results. Suspension preparation involves harvesting the bacterial cells then reconstituting in a 0.5 M saline (NaCl) solution. Some forms of the disclosed methods utilize a culturing method that permits direct application of bacteria onto the matrix and maintains viable bacteria for repeated application. Additionally, results can be drastically improved by using an “immersion” or dipping technique then removing excess bacteria from the matrix through a “squeegee” procedure. Nevertheless, the disclosed methods can be performed using any technique for applying bacteria to the chromatographic matrix, including spray and immersion.

Known thin-layer chromatographic methods involving bacteria do not permit use of acids and bases in the mobile phase, yet acids and bases are common development solvents used in chromatography. These acids and bases are often necessary to achieve adequate component separation of complex mixtures. The disclosed methods and compositions can use a buffering system which not only permits the use of acids and bases in the mobile phase but enhances the quality of the data images by minimizing background interferences cause by environmental contaminants.

Alternative methods discuss potential kit development using a saline solution comprising between 1-3% NaCl wt/vol or about 0.5 M NaCl saline solution. These solutions and methods are not reasonable to sustain viable bacteria. Methods and culture solutions have been discovered that adequately sustain viable bacteria for continued serial transfer and multiple matrix applications. Use of such culture methods and solutions with and in the disclosed methods and compositions provides advantages such as efficiency and convenience. Additionally, the disclosed media can be stored and transported in the kit as a separate or combined dry form thus eliminating or reducing issues associated with the storage and/or transportation of solutions/diluents such as stability, liquid leakage, or breakage. Additionally luminescent bacteria for use in the disclosed methods can be freeze-dried using, for example, 0.1-0.3 g/mL sucrose or sucrose solution. This freeze drying methodology permits the bacteria to be stored indefinitely at 0-8° C. (optimally at approximately 4° C.). These freeze-dried bacteria can also be inoculated and cultured directly into a liquid culture. These freeze-dried bacteria can also be used in the disclosed kits.

Previous detection methods used contact with photographic film to detect and record data. The disclosed methods can use such direct contact of photographic film such as x-ray and Polaroid film but can also collect data indirectly such as with a CCD (charge-coupled device) camera, video imaging, 35 mm camera, and Polaroid documentation system.

The disclosed methods can employ the bioluminescence of microorganisms for bioactivity-specific chromatographic detection by coupling the luminescent microorganisms such as Vibrio fischeri directly to thin-layer chromatography (TLC) or high-performance thin-layer chromatography (HPTLC) [2, 3, 7-13]. Although this technology can be used to analyze single components, it is most beneficial for analyzing complex mixtures. The separation of different components in a complex mixture via thin-layer chromatography allows the presence or absence of one or a plurality of distinct components in the mixture. This technology provides a characteristic pattern and toxicity profile for each compound or mixture analyzed.

Any luminescent bacterium can be used in the disclosed methods and compositions. A particularly useful luminescent bacterium for use in the disclosed methods and compositions is Vibrio fischeri. The observed bioluminescence reflects the metabolic status of the cell and will decrease for cells exposed to toxic substances. Thus a reduction in light emission is a measure of toxicity and can be selectively viewed and quantitated directly on the TLC or HPTLC plate. Typical limits of detection for toxic substances are in the picomol range. Vibrio fischeri is a nonpathogenic, gram-negative species of bacterium that thrives in the marine environment. As this bacterium reaches a crucial cellular density its lux operon expresses the reaction catalyst luciferase. In the presence of O₂ and luciferase, a NADH-reduced riboflavin phosphate (FMNH₂) and a long chain fatty aldehyde are oxidized. The resulting interaction forms an excited yet highly stable intermediate, which decays slowly, resulting in the release of excess free energy in the form of a blue-green light (490 nm) [14-17].

Although the disclosed methods and compositions are described herein primarily by reference to the use of luminescent bacteria, other luminescent biological agents can also be used together with or instead of luminescent bacteria. As used herein, the term “luminescent” biological agent is defined as an organism or an extract of an organism which emits heatless light under appropriate conditions. Most luminescent systems involve the use of molecular oxygen. Luciferin (a pigment) and a specialized form of a luciferase enzyme are included in many luminous organisms and enables these organisms to emit a heatless light in the presence of oxygen. Cypridina is an example of a marine organism which contains the luciferin pigment. For example, Cypridina contains a luciferin which, when reacted with the Cypridina luciferase enzyme in the presence of oxygen, emits a heatless bioluminesence. Vibrio fischeri and Vibrio harveyil contain an enzyme necessary to make light, a well as two reagent compounds (a long-chained aliphatic aldehydes and a vitamin derivative, which is a yellow pigment flavin mononucleotide. In reduced form (i.e., in the presence of oxygen) the pigment glows and allows the organism to emit a heatless light. For example, Cypridina contains a luciferin which, when reacted with the Cypridina luciferase enzyme in the presence of oxygen, emits a heatless bioluminescence. Similarly, fire flies possess a luciferin pigment which in the presence of the firefly luciferase and oxygen, provides a bioluminescence suitable for use in the practice of the present invention. Photobacterium leiognathia is a bacteria which is strongly bioluminescent. All organisms and plants which possess a luciferin/luciferase system would be included among those luminescent biological agents which could be used in the practice of the claimed invention.

Two major subclasses of the luminescent organisms are free living (Vibrio harveyl) and symbiotic (Vibrio fischeri, Photobacterium phosphoreum, Photobacterium leiognathi). Other major bioluminescent organisms include fire flies (Photinus pyralis), crustaceans (Cyridina hilgendorfi), dinoflagellates (Gonyaulax polyhedra, Notiluca militaris), fungi (Omphalia flavida) and the sea pansy (Renilla reniformis). Such organism can be used with and in the disclosed methods and compositions.

TLC and HPTLC separate samples into discrete zones using adsorption and capillary action. These matrices provide a characteristic distribution pattern of compounds that is dependent on sample composition. It is routinely used to support the identity of a compound in a mixture when the rf (retention factor) or migration distance of a compound is compared with the rf of a known compound. In some forms of the method, complex mixtures are first separated by TLC or HPTLC. After compound separation, the matrix plate is coated with bioluminescent bacteria employing a simple dipping procedure, which identifies single toxic compounds as dark zones on a luminescent background. Results can occur within seconds and last until the plate dries, approximately 30 minutes. Data can be documented by direct contact of photographic film such as x-ray and Polaroid film. Data can also be collected indirectly, such as with a CCD camera, video imaging, 35 mm camera, and Polaroid documentation system. Additionally this technology has been designed to be kit compatible.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a method is disclosed and discussed and a number of modifications that can be made to a number of components including the buffer and bacteria are discussed, each and every combination and permutation of buffer or bacteria and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Bacteria

Any suitable bacteria can be sued in and with the disclosed methods and compositions. Useful bacteria include those that can produce a detectable signal where the detectable signal is altered in the presence of compounds, such as toxic or active compounds. The bacteria preferable can produce a visible, colorimetric, fluorescent, luminescent, and/or bioluminescent signal. Useful bacteria can be those capable of producing such signals during normal culture and/or those where production of the signal is made possible or enhanced by including one or more components to the bacteria. For example, some bioluminescent bacteria produce all of the components necessary for producing bioluminescence endogenously during normal growth. As another example, bioluminescence of some bacteria requires or can be enhanced by providing a precursor substrate. A mixture of two or more bacteria can be used in the disclosed methods and compositions.

Useful bacteria include Photobacterium phosphoreum, Photobacterium leiognathi, Vibrio fischeri (for example, ATCC Acc. 7744) and Vibrio harveyi (for example, ATCC Acc. 33843).

B. Culture Medium

The bacteria can be cultured in any suitable manner and using any suitable conditions. The growth and culture of bacteria is generally known and such techniques can be used in and with the disclosed methods and compositions. To ensure the best quality of luminescent bacteria with sustainable viability, the bacteria can be inoculated and maintained in culture medium. Although a variety of media mixtures can be used, the following complex culture medium permits maximum luminescence, growth, and stability that is particularly useful for the disclosed methods. This complex media can contain NaCl (0.5 M), NaH₂PO₄.H₂O (44.2 mM), K₂HPO₄.3H₂O (12.0 mM), MgSO₄.7H₂O (0.8 mM), (NH₄)₂HPO₄ (3.8 mM), 5.0 g/L peptone from casein, 0.50 g/L yeast extract, 6 mL/L 50% aqueous solution of glycerol, and H₂O adjusted to a pH of 7.2±0.2. For optimal results medium can initially be a sterile solution and stored at 4° C. After initial sterilization, media generally can be used for 7 days using standard laboratory practices (non-sterile conditions), although media can be used for longer periods as well.

Culture media can also be stored as combined salts and combined biologicals. Such storage is generally known. Such stored culture media can be used in the disclosed kits, such as the developed TLC-bioluminescence kit. For long term storage the combined salts [NaCl (0.5 M), NaH₂PO₄.H₂O (44.2 mM), K₂HPO₄.3H₂O (12.0 mM), MgSO₄.7H₂O (0.8 mM), (NH₄)₂HPO₄ (3.8 mM)] and the combined biologicals (5.0±0.1 g/L peptone from casein, 0.50±0.01 g/L yeast extract) are best stored in amber bottles under dry conditions, although this is not required. Techniques for the preparation and sterilization of media are well known and can be used with the disclosed media. For example, media can be prepared by combining and dissolving all salts and biologicals in H₂O and adding glycerol (6 mL/L 50% aqueous solution of glycerol, for example). For optimal results medium can initially be a sterile solution and stored at 4° C. After initial sterilization, media generally can be used for 7 days using standard laboratory practices (non-sterile conditions), although media can be used for longer periods as well.

C. Bacteria Storage

Bacteria, such as luminescent bacteria, can be stored for extended periods of time in, for example, a lyophilized or frozen form. Techniques for storage of bacteria and for lyophilization and freeze-drying of bacteria are known and can be used with the disclosed bacteria, methods and compositions. A particularly useful stabilizing agent to lyophilize Vibrio fischeri was determined to be sucrose at a ratio of 0.1-0.3 g sucrose/mL Vibrio fischeri. Particularly useful results can be obtained with Vibrio fischeri or other bacteria used for lyophilizing can be obtained by, for example, culturing the bacteria in complex media described above and/or growing the bacteria to an approximate optical density of 1.7 to 2.5 and/or until luminescencing brightly. Lyophilization of the bacteria can be performed in any suitable vessel, for example flasks or vials.

There are many freeze-drying and freezing techniques/methods known and any can be used or adapted for use with the disclosed bacteria, methods and compositions. The following techniques were determined to be well suited for the disclosed bacteria and methods.

As an example, flask or vial freeze-drying can be performed in the following manner: Transfer freshly prepared liquid culture of Vibrio fischeri or other bacteria to a flask or vial. For best results, the more surface area the quicker the freeze-drying process. Add 0.2 g/mL of the stabilizing agent (sucrose, for example) to the Vibrio fischeri or other bacteria under vigorous shaking for fast and complete dissolution. Solidify the resulting mixture by cooling flask to −35° C. or below. Rotate flask during freezing process to obtain maximum attainable surface area (shell freeze). Freeze dry for 24 hours or until completely dry. Once dry, remove flask or vial and immediately seal. For best results avoid all contact with moisture. Freeze-dried Vibrio fischeri or other bacteria can be stored at 4° C. over extended time, at least 2 years. Lyophilized bacteria in flask can be transferred to smaller vials with caps for storage. Lyophilized bacteria can be included in the disclosed kits.

As an example, frozen bacteria can be prepared in the following manner: Transfer freshly prepared liquid culture of Vibrio fischeri or other bacteria to vial. Add 8-16% glycerol, mix well, cap, and store at approximately −20° C.±2° C. Bacteria are viable and can be used for several months.

D. Bacteria Culture

The bacteria can be cultured in any suitable manner and using any suitable conditions. The growth and culture of bacteria is generally known and such techniques can be used in and with the disclosed methods and compositions. Vibrio fischeri or other bacteria can be inoculated directly into a liquid culture. For example, complete media (preparation described above) can be brought to 20-30° C., room temperature is sufficient. Complete media and 1 vial of bacteria (either lyophilized or frozen) can be transferred to an Erlenmeyer or tissue culture flask. Use of various culture flasks and techniques are known and can be used in the disclosed method. The following are examples of some useful culture techniques. Erlenmeyer flasks can be covered with aluminum foil, cap (must permit sufficient aeration) or plug with non-adsorbent cotton. If using T-flask, such as a T-175 flask, a clean stir bar and cap with sterile filter cap can be added. If using incubator/shaker, incubator/shaker can be set to approximately 28° C. and 120 rpm and transfer covered Erlenmeyer flasks to incubator/shaker. If using stir plate, the flask (either capped tissue culture flask or Erlenmeyer flask with stir bar) can be placed on stir plate and the stir rate can be set to medium-high speed. The culture incubation temperature can vary based on the bacteria used, and optimal and acceptable culture temperatures are generally known. For the use of Vibrio fischeri, a culture temperature of approximately 28° C. is useful, but room temperature will typically suffice. Incubator and stir plate speed and temperatures listed are optimal although these factors can be varied greatly in the disclosed methods; it is only imperative that bacteria be sufficiently aerated and maintained at viable temperatures. Culture flasks can be incubated for any suitable time. As an example, culture flasks can be incubated for 24-30H or until brightly luminescent in a dark room. Culture time can vary (and be varied) with temperature, aeration, and bacteria.

Bacterial cultures can be maintained for extended periods or can be prepared new for each use. For example, bacteria can be serial transferred and continuously used for bioluminescence assays for 2 weeks under sterile conditions and 1 week using standard laboratory practices (non-sterile) with little to no effect on luminescence. Bacteria can also be cultured continuously in, for example, a chemostat. Bacteria can be subcultured to start new culture before using existing culture for assays. This can be accomplished, for example, by bringing complete media to 20-30° C., room temperature is sufficient. Transfer a small aliquot of bacteria (approximately 10-300 μl is optimal for described assay conditions) from luminescent culture into flask. Follow culture procedures described above. Incubate culture flask for 20-24H or until brightly luminescent in a dark room. Bacteria Serial Transfer can be repeated for 5-14 days while maintaining adequate assay properties.

E. Samples and Sample Preparation

Any sample of interest can be used in or with the disclosed methods and composition. The samples can be or can include dietary supplements, natural products, foodstuffs, beverages, waste water, soil samples, pharmaceuticals, pesticides, herbicides, fungicides, insecticides, and heavy metals. The disclosed methods can be used to detect virtually any substance(s) or toxicant(s) in a sample which is capable of inhibiting the signal of the bacteria (such as the luminescence of a luminescent bacterial agent). As examples, the disclosed methods can be used to detect substances such as pesticides, herbicides, heavy metals and their salts, and plant extracts. By way of example, pesticides which can be detected using the disclosed methods include DIAZANON, LINDANE and SEVIN. By way of example, herbicides which can be detected using the disclosed methods include ROUNDUP and WEED-B-GON. Heavy metals which can be detected using the disclosed methods include the identification of mercury, lead, cadmium and their respective salts.

Samples can be prepared and applied to chromatographic matrices in any suitable manner and in any suitable form. In general, samples can be applied to the chromatography plate in any volatile solvent. Although liquid samples can be applied to the plate without preparation, some samples produce higher quality results if processed prior to application. For example, the sugar in high-content sugar samples such as juice or soda can hinder sample separation if not removed prior to chromatography. Sugar can be easily removed via processing the sample through a solid-phase extraction (SPE) cartridge containing a variety of sorbents such as hydrophilic-lipophilic balance (HLB) or diol sorbents. Another example of sample processing that increases chromatography and therefore TLC-bioluminescence quality is the precipitation of proteins in milk or infant formula prior to sample application. Proteins can be easily eliminated through a 1:1 dilution with acetonitrile followed by centrifugation and the resulting supernatant applied to the plate. Although not necessary, sample processing prior to sample application to the chromatography plate can sometimes increase quality. This processing can include any method (solvent partitioning, solid-phase or liquid extraction, precipitation, centrifugation, evaporation, filtering, etc.) known to those skilled in the art of sample preparation. It has also been observed that some analytes display enhanced detection when assayed at a pH between 7 and 9. This can be achieved by adjusting the pH of a sample with an acid or base before applying to the TLC or HPTLC plate.

F. Chromatographic Matrices

The disclosed method can use any suitable chromatographic matrix. The disclosed methods preferably involve thin-layer chromatography and thus suitable matrices include TLC and HPTLC plates. For best results, plates used in assay can be high-performance thin-layer chromatography plates (HPTLC), although paper and thin-layer chromatography plates can also be used. Image quality will also be enhanced if chromatography plates are first “cleaned” with methanol. This can be accomplished by either eluting methanol off the chromatography plate or by soaking the plate in a methanol bath. This and other preparation and treatment techniques and conditions for chromatographic matrices are known and can be used in and with the disclosed methods and compositions.

HPTLC and TLC methods (sample application, development, etc.) employed for the disclosed methods can be standard techniques well known to those skilled in the art. Samples can be applied to the chromatography plate as liquid samples by either hand-spotting or with the use of an automatic spotter. Plates can then be developed using established 1D or 2D chromatography techniques.

All volatile solvents and most acid and bases are compatible with the disclosed methods. After plate development solvent can be removed from plate. This can be done in any manner that does not disrupt the matrix or compounds of interests. For example solvent removal easily occurs in a fume hood or mechanical oven or under a stream of air or nitrogen.

G. Bacteria Coating

Bacteria can be applied to chromatographic matrices using any suitable technique. For example, bacteria can be applied by spraying bacterial cultures or solution onto the matrix, dipping or immersing the matrix in a bacterial culture or solution, pouring a bacterial culture or solution on the matrix, and/or brushing a bacterial culture or solution on the matrix. Bacterial application to the matrix of interest can greatly affect data quality. Therefore, it is preferred that bacteria be applied by immersing the matrix in a bacterial culture or solution. Previous methods prepare a saline bacterial suspension and spray the suspension onto TLC plates. The preparation of the bacterial suspension is time consuming and the spay application produces inferior results. Suspension preparation involves harvesting the bacterial cells then reconstituting in a 0.5 M saline (NaCl) solution. Some forms of the disclosed methods utilize a culturing method that permits direct application of bacteria onto the matrix and maintains viable bacteria for repeated application. Additionally, results can be drastically improved by using an “immersion” or dipping technique then removing excess bacteria from the matrix through a “squeegee” procedure. Nevertheless, the disclosed methods can be performed using any technique for applying bacteria to the chromatographic matrix, including spray and immersion.

As an example of a useful technique for applying bacteria to a matrix, a bacterial culture can be transferred or poured into a chamber for immersing the matrix. The matrix can then be immersed in the bacterial culture. In some forms of the disclosed method, fully luminescent bacteria culture (can use 100% bacteria culture or dilute up to 50% with 2-4% NaCl) can be transferred/poured into an immersion/dipping chamber. HPTLC (or paper or TLC plate) can then be immersed into the bacteria. This can be done with an automatic immersion device or hand-dipped. For best results matrix should be dipped in one attempt at a steady rate.

Optimal results can be achieved by removing excess bacteria from plate before data detection. Thus, in some forms of the disclosed methods, a “squeegee” effect, such as by use of a squeegee device, can be used to remove excess bacteria from the chromatographic matrix. It has been discovered that removal of excess bacteria from the chromatographic matrix provides more reliable and consistent results and readouts of luminescence in the disclosed methods. Generally, the squeegee effect can be accomplished by moving the matrix and a linear edge or surface relative to each other in the plane of the matrix. The linear edge or surface can be provided in any way and using any suitable structure. For example, a squeegee device; a material with an edge, such as a ruler, straight edge, sheet; a solid or hollow object with a turn or corner, such as a bar with a polygonal section; a solid or hollow object with both a curve and a linear dimension, such as a roller or circular bar. The squeegee effect can be achieved, for example, by creating a narrow gap between a squeegee device and the matrix and moving the squeegee device across the matrix such that the excess culture liquid is pushed ahead of the leading edge or surface of the squeegee device. To remove excess bacteria, the matrix, such as the matrix side of the TLC plate, can be gently squeegeed. Care should be given not to disturb TLC/HPTLC matrix. Although the “squeegee” process can be done with many devices that will remove bacteria without disturbing the matrix, the best results are obtained using a squeegee device. After removing excess bacteria, the chromatography plate is ready for detection (such as TLC-bioluminescence detection).

H. Bacteria Viability

Bacteria can remain viable in dipping/immersion chamber for greater than 2 hours if aerated. This is best accomplished by aspirating air into chamber at least every 5-20 minutes.

I. Buffers and pH Adjusting Substances

The disclosed method can be made compatible with the use of a wide variety of chromatographic solvents and acids and bases used in chromatographic solvents by using a buffers and/or pH adjusting substances with the bacteria. It has been discovered that use of certain buffers and pH adjusting substances, such as HEPES buffer, with the bacteria eliminates or reduces negative effects the chromatographic solvents and acids and bases would otherwise have on the bacteria. The use of such buffers and pH adjusting compounds allows the disclosed methods and compositions to use virtually any desired chromatographic solvent or acid or base used in chromatographic solvents.

The disclosed methods can be transformed to be acid/base compatible by buffering or adjusting the pH of the bacteria and/or matrix before coating. This is best done using a biological buffering system that buffers in the 7.5±1 pH range. Nevertheless, any suitable buffer or pH adjusting substance can be used in and with the disclosed method. A particularly useful buffing system was determined to be HEPES [N-(2-Hydroxyethyl)piperazine-N′-(2-ethanesulfonic acid)] buffer. The buffers and pH adjusting substances can be used in any appropriate amount or concentration. This generally can be determined using known principles based on the identity and concentration of acids and bases used in chromatography. As an example, HEPES can be used in the 0.2-0.5 M range. It is useful if the buffer is added or applied before the bacteria are applied to the matrix. For example, buffer can be transferred to fully luminescent bacteria culture before assay. After complete dissolution of the buffer, bacteria can be applied to the matrix and the detection can be performed. Buffered or pH adjusted bacteria can remain viable for some period of time if properly aerated. For example, HEPES buffered bacteria can remain viable in dipping/immersion chamber (or in flask) if properly aerated for greater than 2 hours.

J. Detection

The effect of compounds on bacteria coating chromatographic matrices can be assessed in any suitable manner. Useful detection can be accomplished by observing a change, usually a decrease, in a signal produced by the bacteria after contact with the matrix. In useful forms of the methods, compounds affect or reduce the viability of the bacteria. The reduced viability causes the bacteria to reduce or cease producing the signal. For example, many bioluminescent bacteria only luminesce when in a health state. Contact with toxic compounds affects the health of the bacteria enough to cause them to stop luminescing. In the disclosed method, the effect of the toxic or active compounds is localized to the site on the matrix where the compounds migrated during chromatography.

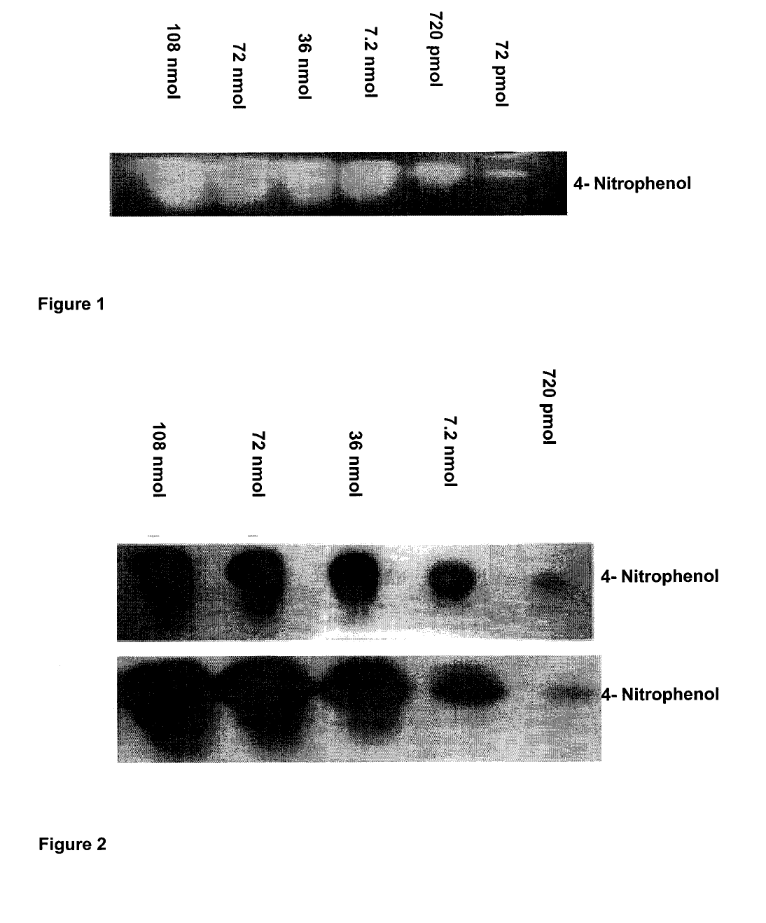

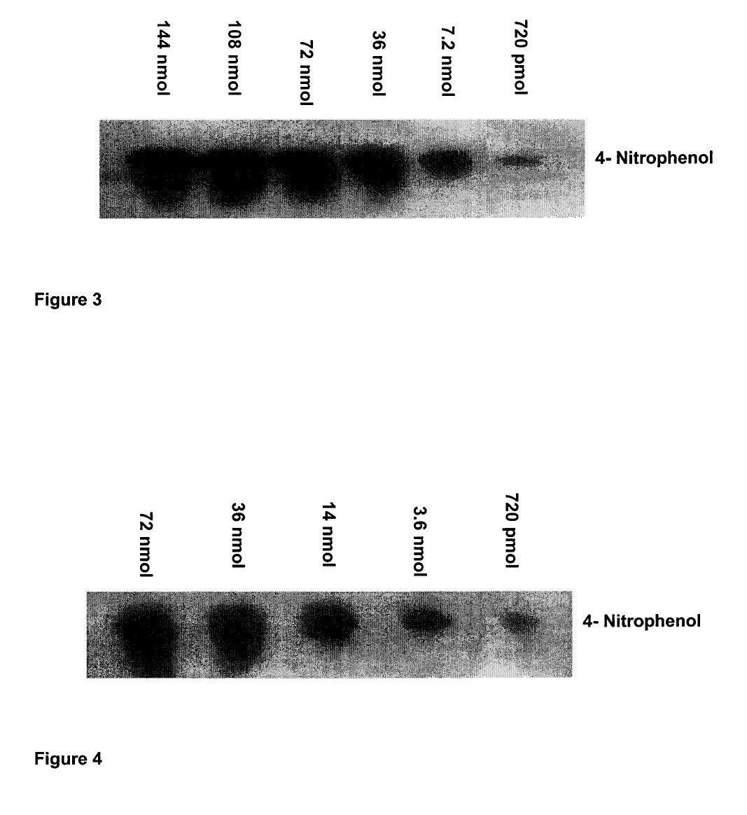

Bacteria coated plates can be viewed and documented by direct contact of photographic film such as x-ray and Polaroid film. This is best accomplished by covering the bacteria coated chromatography plate with a thin film of plastic wrap. Film can then be placed in direct contact with the plastic wrap thus eliminating bacterial interference on the film. Data can also be collected indirectly such as with a cooled CCD camera, video imaging, 35 mm camera, and Polaroid documentation system (FIGS. 1-4). Results are proportional to luminescence inhibition and are concentration dependent. Therefore data can be quantitated by comparing zone contrast which is easily done with a variety of equipment/software such as a grey scale guide or densitometry software.

K. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detection of compound, the kit comprising bioluminescent bacteria and buffer, bioluminescent bacteria and TLC or HPTLC plates, buffer and TLC or HPTLC plates, or bioluminescent bacteria, buffer and TLC or HPTLC plates. The kits also can contain a squeegee device, for example.

L. Mixtures

Disclosed are mixtures formed by performing or preparing to perform the disclosed method. For example, disclosed are mixtures comprising bioluminescent bacteria and buffer, bioluminescent bacteria and TLC or HPTLC plates, buffer and TLC or HPTLC plates, or bioluminescent bacteria, buffer and TLC or HPTLC plates.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

M. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

N. Data Structures and Computer Control

Disclosed are data structures used in, generated by, or generated from, the disclosed method. Data structures generally are any form of data, information, and/or objects collected, organized, stored, and/or embodied in a composition or medium. An image of a chromatograph stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, detection of toxic compounds in complex mixtures. Other uses are disclosed, apparent from the disclosure, and/or will be understood by those in the art.

### EXAMPLES

### A. Example 1

### Uncaria tomentosa (Powdered Cat's Claw Extract)

Sample preparation: Cat's Claw samples were prepared by adding 122.11 mg powdered and 108.64 mg dried aqueous extract of Cat's Claw herb to separate 10 mL volumetric flasks. Methanol (2.5 mL) was added to each flask and resulting solutions sonicated for 15 minutes. Samples were then cooled to room temperature and flasks filled to volume with diluent. Flasks were stoppered, mixed well, and decanted into a scintillation vials. The vials were centrifuged for 1 hour. An aliquot of the supernatant was filtered into an HPLC vial using a 0.45 μm PTFE syringe filter.

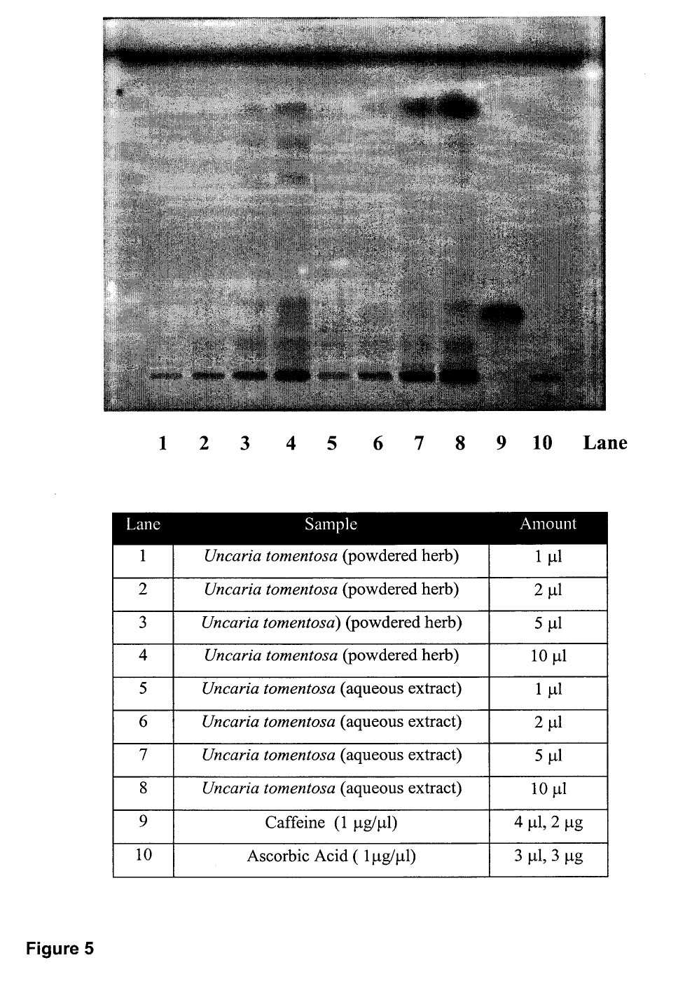

HPTLC-bioluminescence: One—ten μL of resulting samples (powdered herb extract and aqueous extract), 4 μg caffeine (positive control), and 3 μg ascorbic acid (negative control) were applied to a pre-washed (methanol elution) 10×10 cm Merck HPTLC F₂₅₄ plate at y=8 mm using the band spray application mode of an automatic spotter. After sample application the plate was dried for 15 minutes in a fume hood. The plate was then developed to 70 mm in a pre-equilibrated (30 minutes) 10×10 flat bottom chamber with ethyl acetate-isopropyl alcohol-ammonia (100:2:1). Developed plate was dried in a mechanical oven at 40° C. for 2 h prior to bacterial immersion. Plate was coated with HEPES buffered (0.3 M) luminescent Vibrio fischeri using an automatic immersion device (CAMAG, 200 ml). Excess bacteria was removed from the plate using a squeegee device and images were immediately recorded over a 10 minute period using an exposure time of 2 minutes with a cooled (−30° C. absolute) CCD camera (Fluorochem® 8900, Alpha Innotech) and dark box. Results can be viewed in FIG. 5.

### B. Example 2

### Actaea racemosa/Actaea pachypoda (Black and White Cohosh)

Sample preparation: Plant materials, Actaea racemosa (3.17 g rhizome) and Actaea pachypoda (1.11 g leaf) were extracted with 95% aqueous ethyl alcohol (AAPER control # SD-03-600016) (15 ml and 5 ml respectively). Extracts were sonicated for 10 min before filtration with a 0.8 μm Versapor® membrane syringe filter. Each extraction procedure was repeated 3 times and resulting filtrates combined. Each combined filtrate was then concentrated to dryness using rotovapory evaporation yielding Actaea racemosa (153.8 mg rhizome) extract and Actaea pachypoda (86.4 mg leaf) extract. Extracts were reconstituted in a known amount of CH₃OH, an aliquot removed and diluted to 10 mg/ml for analysis. Aliquots from each stock solution were combined to yield 0% (500 μl), 25% (125 μl/375 μl), 50% (375 μl/375 μl), and 75% (375 μl/125 μl) of Actaea pachypoda leaf extract in Actaea racemosa rhizome extract. Resulting solutions were examined with the BioLuminex assay.

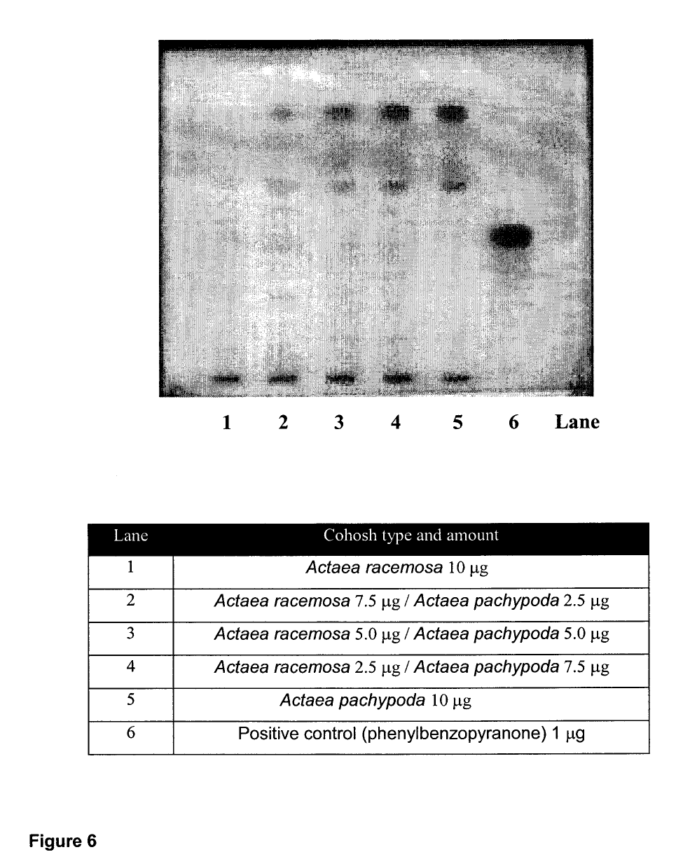

HPTLC-bioluminescence: Resulting solutions were diluted to 5 mg/ml with CH₃OH and 2 μl of each sample and 1.0 μg of positive control (phenylbenzopyranone) (TCI) was applied in CH₃OH (EMD) to a pre-washed (methanol bath) 10×10 cm Merck HPTLC_{F254} plate at y=8 mm using the band spray application mode of an automatic TLC spotter. After sample application the plate was dried for 15 minutes in a fume hood. The plate was then developed to 70 mm in a pre-equilibrated (30 minutes) 10×10 flat-bottom chamber with toluene-ethyl acetate-formic acid (5:3:2) (Sigma Aldrich). Developed plate was then dried in a mechanical oven at 40° C. for 3 h prior to bacterial immersion. Plates where coated with Tris buffered (0.3 M) luminescent Vibrio fischeri using an automatic immersion device (CAMAG, 200 ml). Excess bacteria was removed from the plate using a squeegee device and images were immediately recorded over a 10 minute period using an exposure time of 60 seconds with a cooled (−30° C. from ambient) CCD camera (SynGene) and dark box. Results can be viewed in FIG. 6.

### C. Example 3

### Infant Formula/Strychnine

Sample preparation: Infant formula (Similac Infant Formula with Iron-Powder, Abbott Laboratories) was prepared by dissolving 1.0607 g formula into 7.5 ml H₂O resulting in a 141.4 mg/ml mixture (CDXA-019-131-1, Infant formula standard). 47.6 mg of strychnine (Sigma S0532) was dissolved into 4.76 ml of prepared infant formula (CDXA-091-137-1). 4.5 ml aliquot of sample was combined with 4.5 ml of ACN. The mixture was vortexed for 30 sec before centrifugation at 6400 g for 8 min. The resulting supernatant was removed (CDXA-019-137-2) and analyzed by BioLuminex technology.

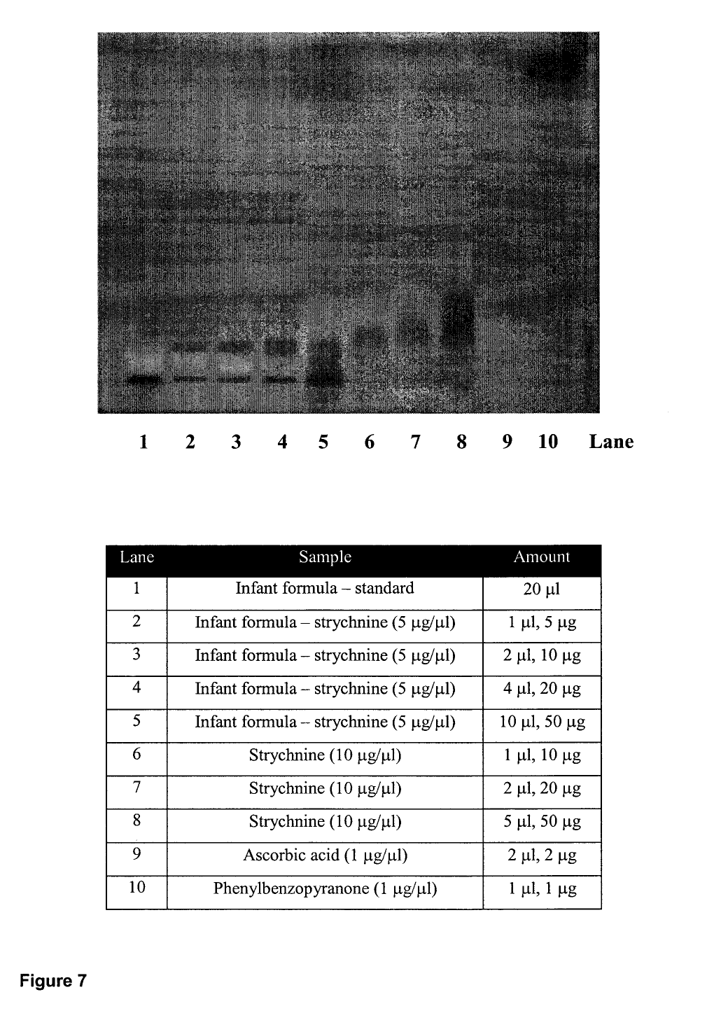

HPTLC-bioluminescence: One—ten μl of resulting supernatant, 1.0 μg positive control (phenylbenzopyranone) (TCI), and 2 μg ascorbic acid (negative control) were applied in CH₃OH (EMD) to a pre-washed (methanol elution) 10×10 cm Merck HPTLC_{F254} plate at y=8 mm using the band spray application mode of an automatic TLC spotter. After sample application the plate was dried for 15 minutes in a fume hood. The plate was then developed to 70 mm in a pre-equilibrated (30 minutes) 10×10 twin trough chamber with toluene-ethyl acetate-formic acid (5:3:2) (Sigma Aldrich). Developed plate was then dried in a mechanical oven at 40° C. for 2 h prior to bacterial immersion. Plate was coated with HEPES buffered (0.3 M) luminescent Vibrio fischeri using an automatic immersion device (CAMAG, 200 ml). Excess bacteria was removed from the plate using a squeegee device and images were immediately recorded over a 10 minute period using an exposure time of 2 minutes with a cooled (−30° C. absolute) CCD camera (Fluorchem® 8900, Alpha Innotech) and dark box. Results can be viewed in FIG. 7.

### D. Example 4

### Orange Juice/Strychnine

Sample preparation: Orange juice spiked with strychnine (83.8 mg strychnine dissolved in 16 mL orange juice) and un-spiked orange juice were each first filtered through a small bed of glass wool packed into a 30 mL plastic syringe. Two Strata-X HLB SPE cartridges were cleaned and conditioned with 8 mL acetonitrile, 8 mL 80% methanol w/1% ammonia, and 8 mL methanol w/3% acetic acid. Cartridges were then equilibrated with 24 mL of Millipore water before loading 8 mL spiked and un-spiked juice (flow though collected as FTE). Cartridges were then washed with 5 mL Millipore water (wash), 5 mL 80% methanol w/1% ammonia (elution 1), 5 mL methanol w/3% acetic acid (elution 2) and 5 mL 25% dichloromethane/isopropyl alcohol (elution 3). The resulting wash fractions were further processed through diol SPE cartridges. Two diol SPE cartridges were first cleaned and conditioned with 3 mL methanol w/3% acetic acid and 6 mL acetonitrile before loading 6 mL of each wash (diluted with acetonitrile 1:1). Cartridges were then washed with 2 mL acetonitrile (Diol/Wash) and 2 mL Millipore water (Diol/Elution).

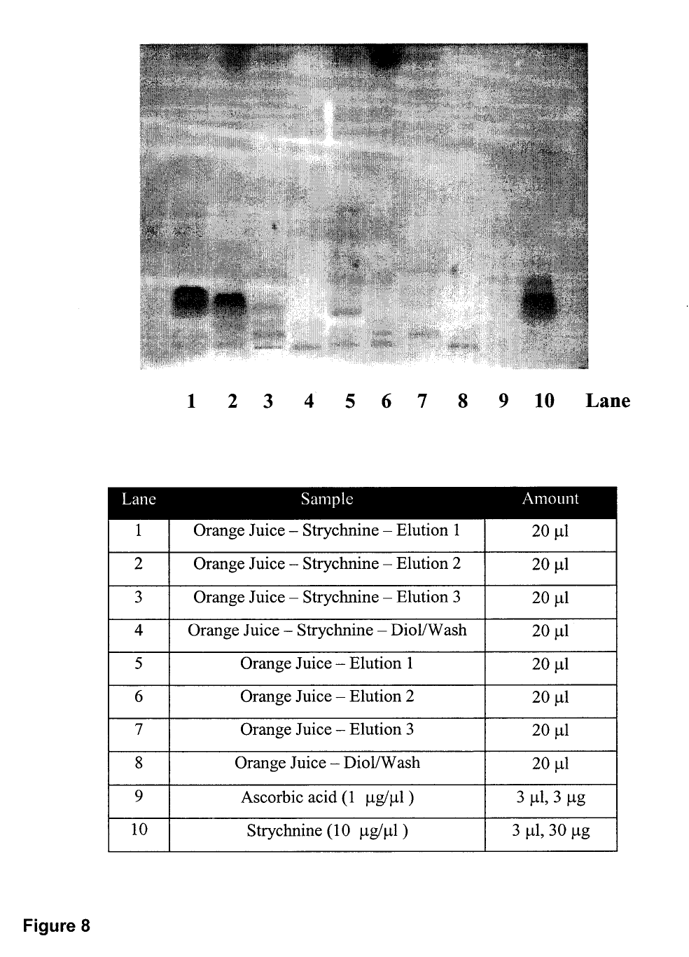

HPTLC-bioluminescence: Resulting fractions (20 μL), 30 μg positive control (strychnine), and 2 μg ascorbic acid (negative control) were applied in CH₃OH (EMD) to a pre-washed (methanol elution) 10×10 cm Merck HPTLC_{F254} plate at y=8 mm using band spray application. After sample application the plate was dried for 15 minutes in a fume hood. The plate was then developed to 70 mm in a pre-equilibrated (30 minutes) 10×10 flat bottom chamber with EtoAc-CH₃OH—H₂O-formic acid (50:2:3:5). Developed plate was then dried in a mechanical oven at 40° C. for 2 h prior to bacterial immersion. Plate was coated with HEPES buffered (0.3 M) luminescent Vibrio fischeri using an automatic immersion device (CAMAG, 200 ml). Excess bacteria was removed from the plate using a squeegee device and images were immediately recorded over a 10 minute period using an exposure time of 2 minutes with a cooled (−30° C. absolute) CCD camera (Fluorchem® 8900, Alpha Innotech) and dark box. Results can be viewed in FIG. 8.

### E. Example 5

### Cola Drink/Monofluoroacetic Acid (Sodium Fluoroacetate)

Sample Preparation: Sodium fluoroacetate spiked cola (53.4 mg of sodium fluoroacetate dissolved in 10 mL Cola drink) and un-spiked cola drink were each acidified to pH 0.5 with 0.25 mL of 37% hydrochloric acid. Two Strata-X HLB SPE cartridges were cleaned and conditioned by passing 8 mL acetonitrile, 8 mL 80% methanol w/1% ammonia, and 8 mL methanol w/3% acetic acid. Cartridges were then equilibrated with 24 mL Millipore water before loading 8 mL of each solution and collecting flow through (FTE). Cartridges were then washed with 3 mL of Millipore water (wash), 5 mL methanol (elution 1), 5 mL acetonitrile (elution 2) and 5 mL 25% dichloromethane/isopropyl alcohol (elution 3). 0.1 N sodium hydroxide was added to 1 mL aliquots of the spiked soda and the elution 1 sample to adjust the pH into the 7 to 9 range.

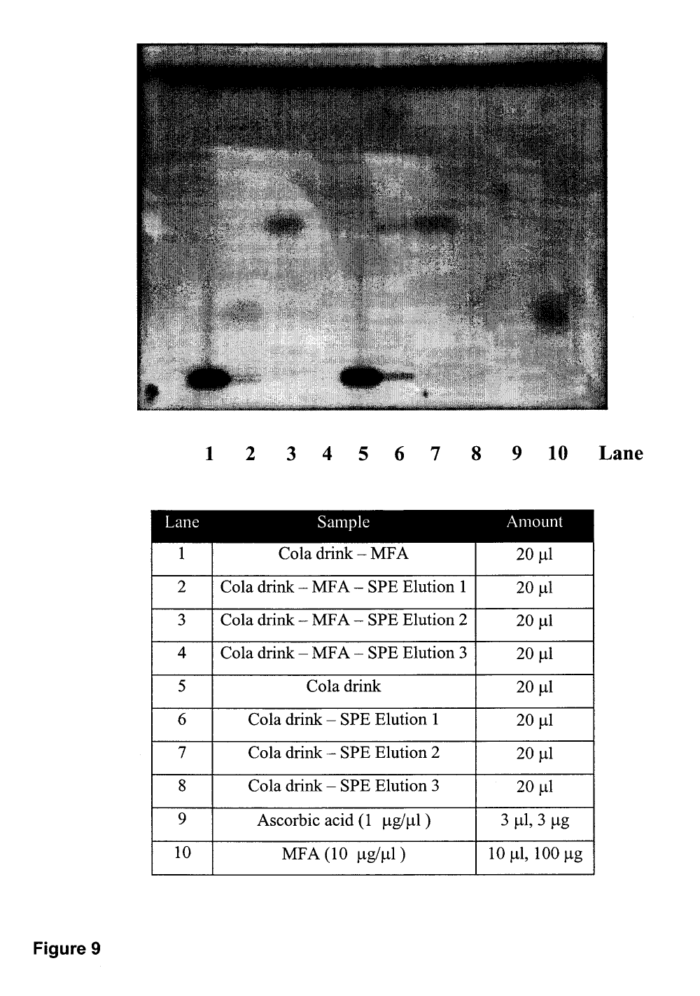

HPTLC-bioluminescence: Resulting fractions (20 μL), 100 μg positive control (sodium fluoroacetate), and 3 μg ascorbic acid (negative control) were applied in CH₃OH (EMD) to a pre-washed (methanol elution) 10×10 cm Merck HPTLC_{F254} plate at y=8 mm using band spray application. After sample application the plate was dried for 15 minutes in a fume hood. The plate was then developed to 70 mm in a pre-equilibrated (30 minutes) 10×10 flat bottom chamber with EtoAc-CH₃OH—H₂O-formic acid (50:2:3:5). Developed plate was then dried in a mechanical oven at 40° C. for 2 h prior to bacterial immersion. Plates where coated with HEPES buffered (0.3 M) luminescent Vibrio fischeri using an automatic immersion device (CAMAG, 200 ml). Excess bacteria was removed from the plate using a squeegee device and images were immediately recorded over a 10 minute period using an exposure time of 2 minutes with a cooled (−30° C. absolute) CCD camera (Fluorchem® 8900, Alpha Innotech) and dark box. Results can be viewed in FIG. 9.

### F. Example 6

### Tap Water Spiked with a Steroid, Heavy Metal, and Pesticide

Sample Preparation: Standard solutions (1 mg/ml) of arsenic oxide, Metolachlor, and 4-androstene-3,17-dione were prepared. Arsenic oxide (15.11 mg) was dissolved into 15.11 mL Millipore water. The resulting solution was then sonicated for approximately 10 minutes and heated for 30 minutes at 50° C. Metolachlor (14.41 mg) was dissolved into 14.41 mL Millipore water and 4-androstene-3,17-dione (9.53 mg) was dissolved into acetone. The resulting solutions were thoroughly mixed by vortexing. A spiked solution of tap water was then prepared by adding 0.4 mL of each standard solution (1 mg/mL) and diluting to 5 mL with tap water. The resulting solution was thoroughly mixed by vortexing. Aliquots of the spiked tap water and the unspiked tap water were transferred to amber HPLC vials for analysis.

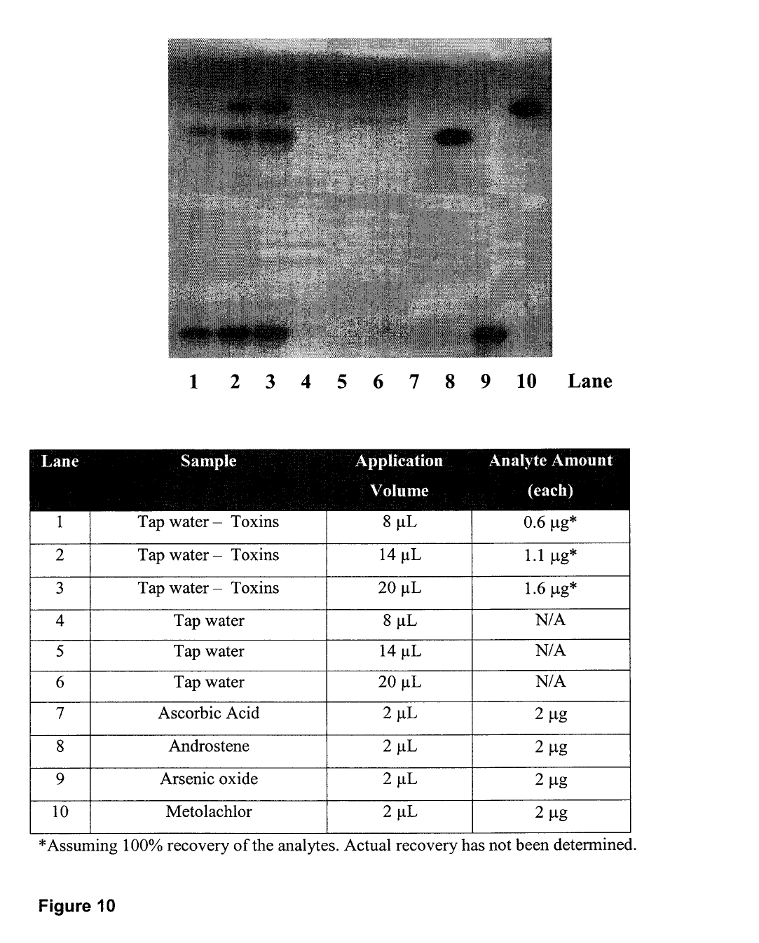

HPTLC-bioluminescence: The samples and the arsenic standard were applied in water to a pre-washed (methanol elution) 10×10 cm Merck HPTLC silica gel 60 F₂₅₄ plates (Lot # OB494661) via a CAMAG Automatic TLC Spotter 4 (ATS-4) at y=8 mm using band spray application set to a water application mode. The remaining standards were applied in methanol to the same plate using the band spray application set to a methanol application mode. After sample application the plate was air dried for 15 minutes. The plate was developed to 70 mm using a mobile phase of toluene:ethyl acetate: formic acid:water (4:8:1.1:0.2) in a pre-equilibrated (30 minutes) 10×10 cm ridged bottom chamber. Post development the plate was dried in a mechanical oven at 40° C. for 1.5 hours. Plates where coated with HEPES buffered (0.3 M) luminescent Vibrio fischeri using an automatic immersion device (CAMAG, 200 ml). Excess bacteria was removed from the plate using a squeegee device and images were immediately recorded over a 10 minute period using an exposure time of 2 minutes with a cooled (−30° C. absolute) CCD camera (Fluorchem® 8900, Alpha Innotech) and dark box. Results can be viewed in FIG. 10.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a compound” includes a plurality of such compounds, reference to “the compound” is a reference to one or more compounds and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Goodall, R. R. and A. A. Levi, Nature (London), 1946.158: p. 675-676.
2. Fischer, R. and H. Lautner, On the Paper Chromatograohic Detection of Penicillin Preparations. Arch. Pharm., 1961. 294(66): p. 1-7.
3. Nicolaus, B. J., C. Coronelli, and A. Binaghi, Microbiological Determination of Antibiotics by Thin Layer Chromatograms. Experientia, 1961. 17(473): p. 473-4.
4. Choma, I. The Use of Thin-Layer Chromatography with Direct Bioautography for Antimicrobial Analysis. LC-GC Europe 2005 Sep. 1, 2005 [cited 2005 Sep. 20, 2005].
5. Nicolaus, B. J., C. Coronelli, and A. Binaghi, “Applicazione agli antibiotici delta cromatografia su strato sottile”. Farmaco Ed. Prat., 1961. 16(349).
6. Merck KGaA, D. G. ChromBiodip® Antibiotics. Apr. 26, 2004 [cited 2005 Sep. 20]; Available from: http://chrombook.merck.de/chrombook/ip_chrombook-documents/out-chrombook/out-1-1-7-4.htm.
7. Weisemann, C., et al., Analytical Method for Investigating Mixtures of Toxic Compounds., E. P. Office, Editor. 1998, Bayer AG: Germany. p. 7.
8. Eberz, G., et al., Bioactivity Screening by Chromatography Bioluminescence Coupling. Chromatographia, 1996. 43: p. 5-9.
9. Becvar, J. E. and L. Becvar, E., Kit for the Isolation, Identification, and Quantitation of Toxicants., U.S.P. Office, Editor. 2002, Board of Regents, The University of Texas System: United States. p. 27.
10. Becvar, J. E. and L. E. Becvar, Luminous Bacteria and Methods for the Isolation, Identification, and Quantitiation of Toxicants., U.S.P. Office, Editor. 2000, Board of Regents, The University of Texas System: United States. p. 32.
11. Becvar, J. E. and L. E. Becvar, Luminous Bacteria and Methods for the Isolation, Identification, and Quantitation of Toxicants., U.S.P. Office, Editor. 2004: United States. p. 28.
12. Weisemann, C., et al., Analytical Process for Testing Mixtures for Toxic Constituents., U.S.P.a.T. Office, Editor. 2001, Bayer Aktiengesellschaft. p. 6.
13. Weins, C. and H. Jork, Toxicological Evaluation of Harmful Substances by In Situ Enzymatic and Biological Detection by High-Performance Thin-Layer Chromatography. Journal of Chromatography A, 1996. 750(1-2): p. 403-407.
14. Meighen, E. A., Bacterial Bioluminescence: Organization, Regulation, and Application of the Lux Genes. FASEB, 1993. 7: p. 1016-1022.
15. Lee, J., et al., eds. The Mechanism of Bacteria Bioluminescence. In Chemistry and Biochemistry of Falvoenzymes., ed. F. Muller. Vol. II. 1990, CRC Press: Boca Raton, Fla. 109-151.
16. Engebrecht, J., K. Nealson, and M. Silverman, Bacerial Bioluminscence: Isolation and Genetic Analysis of Functions from Vibrio fischeri. Cell, 1983. 32(3): p. 773-781.
17. Hastings, J. W. and K. H. Nealson, Bacterial Bioluminescence. Annu Rev Microbiol, 1977.31: p. 549-595.

We claim:

1. A method comprising
bringing into contact luminescent bacteria, a pH adjusting component and a thin-layer chromatography matrix, wherein the chromatography matrix comprises a sample separated by thin-layer chromatography, and
detecting inhibited luminescence.

2. The method of claim 1, wherein the luminescent bacteria are brought into contact with the chromatography matrix by applying the bacteria to the chromatography matrix and using a squeegee effect to remove excess bacteria from the chromatography matrix.

3. The method of claim 2, wherein the squeegee effect is achieved using a squeegee device.

4. The method of claim 2, wherein the bacteria are applied by immersing the chromatography matrix is a liquid comprising the bacteria.

5. The method of claim 4, wherein the liquid further comprises the pH adjusting compound.

6. The method of claim 1, wherein the pH adjusting component is a buffer.

7. The method of claim 6, wherein the buffer buffers in the 7.5±1 pH range.

8. The method of claim 6, wherein the buffer is in the 0.2-0.5 M concentration range.

9. The method of claim 6, wherein the buffer is HEPES [N-(2-Hydroxyethyl)piperazine-N′-(2-ethanesulfonic acid)]buffer.

10. The method of claim 1, wherein the luminescence inhibition is recorded with x-ray or Polaroid film, cooled CCD camera, video imaging, 35 mm film, or Polaroid photo documentation system.

11. The method of claim 1, wherein the luminescent bacteria comprises Vibrio fischeri.

12. The method of claim 1, wherein the sample comprises dietary supplements, natural products, foodstuffs, beverages, waste water, soil samples, pharmaceuticals, pesticides, herbicides, fungicides, insecticides, heavy metals, or a combination.

13. The method of claim 1, wherein detection of inhibited luminescence indicates the presence of an active compound in the chromatography matrix at the site of the inhibited luminescence.

14. A method comprising
bringing into contact luminescent bacteria and a thin-layer chromatography matrix, wherein the chromatography matrix comprises a sample separated by thin-layer chromatography, wherein the luminescent bacteria are brought into contact with the chromatography matrix by applying the bacteria to the chromatography matrix and using a squeegee effect to remove excess bacteria from the chromatography matrix, and
detecting inhibited luminescence.

15. The method of claim 14, wherein the squeegee effect is achieved using a squeegee device.

16. The method of claim 14, wherein the bacteria are applied by immersing the chromatography matrix is a liquid comprising the bacteria.

17. A kit comprising luminescent bacteria and a pH adjusting component.

18. The kit of claim 17 further comprising a thin-layer chromatography matrix.

19. The kit of claim 17 further comprising a squeegee device.

20. The kit of claim 17, wherein the pH adjusting component is a buffer.

21. The kit of claim 20, wherein the buffer buffers in the 7.5±1 pH range.

22. The kit of claim 20, wherein the buffer is in the 0.2-0.5 M concentration range.

23. The kit of claim 20, wherein the buffer is HEPES [N-(2-Hydroxyethyl)piperazine-N′-(2-ethanesulfonic acid)]buffer.

24. The kit of claim 17, wherein the luminescent bacteria comprises Vibrio fischeri.

25. The kit of claim 17 further comprising media culture materials.

26. The kit of claim 25, wherein the media culture materials are stored as a combined dry form.

27. The kit of claim 17, wherein the luminescent bacteria are stabilized by lyophilization in the presence of sucrose at a ratio of 0.2 g±0.1 sucrose/mL.
